(19) United States
(12) Patent Application Publication
(10) Pub. No.: US 2009/0270911 A1
(43) Pub. Date: Oct. 29, 2009

(54) Vessel Sealing Device and Method of Using Same

(76) Inventor: John I. Shipp, Atlantic Beach, FL (US)

Correspondence Address: Michael L. Leetzow, Esq.;Michael L. Leetzow, P.A., 5213 SHORELINE CIRCLE, SANFORD, FL, 32771, US

(21) Appl. No.: 12/108,752

(22) Filed: Apr. 24, 2008

(51) Int. Cl. A61B 17/122; A61B 17/04

(52) U.S. Cl. 606/213; 606/232

(57) ABSTRACT

A sealing device for sealing openings in blood vessels has a first and second sealing elements. The first element is preferably fixed attached to a rigid shaft. The second sealing element is slidingly movable relative to the rigid shaft in only one direction. The first and second sealing elements directly engage the walls of the blood vessel. One embodiment has a two portion shaft and one of the portions can be removed after the hole in the blood vessel is sealed.

### BACKGROUND OF THE INVENTION

1. Field of the Invention

The present invention relates generally to a sealing device for the closure of puncture holes in blood vessels and, in particular, to a sealing device for puncture holes in blood vessels where a first element is in a predetermined relationship to a rigid shaft.

2. Technical Background

For many diagnostic and interventional procedures it is necessary to access arteries or veins. Vessel access is accomplished either by direct vision or percutaneously. In either case, the target vessel is punctured with a hollow needle containing a tracer wire. When the intravascular positioning of the tracer wire has been verified, the hollow needle is removed leaving the tracer wire. Next, a sheath containing a dilator is pushed in over the tracer wire. The dilator enlarges the puncture opening to facilitate the insertion of the larger diameter sheath into the blood vessel. The sheath usually consists of a hollow tube with an open distal end and a hemostatic valve at a proximal end, which remains outside the body and vessel. The hemostatic valve is made of a compliant material and is designed in such a way as to allow devices such as catheters to be inserted and withdrawn from the vessel with minimal blood loss. After the sheath has been inserted into the blood vessel, the dilator is removed leaving a clear passageway in the sheath for the catheter. The sheath is removed from the blood vessel after the procedure is finished resulting in bleeding at the puncture site that must be staunched.

Traditionally, pressure is applied to the puncture site to allow time for the blood to clot thereby stopping the bleeding. Depending on the amount of anticoagulants that may have been administered to the patient during and prior to the procedure, the time pressure must be maintained varies from 15 minutes to more than an hour. Once bleeding has stopped, a pressure bandage is placed over the site of the puncture in an attempt to protect the integrity of the clot. The pressure bandage must remain in place for some time, usually from 8 to 24 hours. During this period of time the patient must remain in bed, sometimes requiring an overnight hospital stay.

To shorten the length of time required for the patient to become ambulatory and to lessen complications sometimes arising from the traditional method, several closure devices have been developed. One such device includes an absorbable collagen plug cinched down against an absorbable intervascular anchor via an absorbable suture. The anchor has an elongated rectangular shape that requires it to be inserted into the puncture wound with its longitudinal axis parallel to the sheath axis. This requires it to be rotated ninety degrees after insertion so that blood flow obstruction is minimized. The long dimension of the anchor is thus larger than the cannula inside diameter (ID) and the width is smaller than the ID. The collagen plug is in an elongated state prior to deployment and is forced into a ball shape via a slipknot in the suture, which passes through the collagen, and a tamper that applies a distal force to it. The anchor acts as a support for the suture cinch which forces the collagen ball shape up against the exterior vessel wall and the anchor. Blood flow escaping around the anchor is slowed down and absorbed by the collagen material and thus forms a clotting amalgamation outside the vessel that is more stable than the traditional method of a stand alone clot. The added robustness of the amalgamation clot allows earlier ambulation of the patient.

The device raises several issues. Its use requires removing the catheter sheath and replacing it with a custom sheath prior to deploying the device, resulting in addition blood loss. The tamping force used to deploy the collagen against the anchor is left to the surgeon's feel sometimes resulting in inadequate deployment and other times resulting in the collagen being pushed through the puncture wound, into the vessel along with the anchor. The former results in excessive bleeding with the potential for painful hematoma and the latter requires a surgical procedure to remove the device from the vessel lumen. In addition, the absorption rate of the suture, the collagen, and the anchor may be different owing to the fact that they are formed from different materials, sometimes resulting in the detachment of the anchor, which moves freely in the blood stream and becomes lodged in the lower extremities of the body, again requiring surgical removal.

Additionally, the prior art device does not have the capability of use of a tracer wire, thus backup methods cannot be easily employed in case of device or deployment method failure. It is worth noting that the device relies on clotting and is not a true vessel seal.

It would be desirable therefore to provide a vessel-sealing device that actually seals the vessel and does not rely on the clotting of the blood and allows the use of a tracer wire.

### SUMMARY OF THE INVENTION

Disclosed herein is a sealing device for sealing an opening in the wall of a blood vessel, the blood vessel having an interior wall surface, and exterior wall surface, and a lumen, that includes a first sealing element for placing inside the lumen of the blood vessel and to engage the interior wall surface thereof, a rigid shaft fixedly attached to the first sealing element in a predetermined configuration, the rigid shaft having a length sufficient to extend through the opening of the blood vessel and at least a portion of any overlying tissue, and a second sealing element, the second sealing element slidingly movable relative to the first sealing element in only one direction along the rigid shaft to engage the outside surface of the blood vessel and position the first sealing element against the interior surface of the blood vessel to seal the opening in the blood vessel.

In some embodiments, the first sealing element and the rigid shaft are one integral element.

In some embodiments, the first sealing element and the rigid shaft are perpendicular to one another.

In some embodiments, the rigid shaft has a first portion and a second portion, the two portions are removably connected to one another.

In some embodiments, the sealing device also includes a collagen plug that is disposed on a proximal side of the second sealing element.

In another aspect, a method of sealing an opening in the wall of a blood vessel is disclosed, the blood vessel has an interior wall surface, and exterior wall surface, and a lumen, the method includes providing a sealing device comprising a first sealing element, a rigid shaft fixedly attached to the first sealing element in a predetermined configuration, the rigid shaft having a length sufficient to extend through the opening of the blood vessel and at least a portion of any overlying tissue, and a second sealing element, the second sealing element slidingly movable relative to the first sealing element in only one direction along the rigid shaft, providing an elongated sheath, the sheath capable of receiving the sealing device into an interior opening extending along the sheath, inserting the sheath into the opening in the wall of the blood vessel, inserting the sealing device into the opening in the wall of the blood vessel through the sheath such that at least the first sealing element extends beyond a distal end of the sheath and is disposed in the lumen of the blood vessel, moving the sheath in a proximal direction and out of the blood vessel opening such that the first sealing element engages at least a portion of the interior wall surface of the blood vessel, slidingly moving the second element along the rigid shaft and towards the first sealing element such that the second element engages the exterior wall surface of the blood vessel, thereby pinching the blood vessel between the first and second sealing elements to seal the opening in the blood vessel, and removing the sheath from the sealing device.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is an elevational view of a first embodiment of a vessel sealing device according to the present invention;

FIG. 1A is an elevational view of a second embodiment of a vessel sealing device according to the present invention;

FIG. 2 is a view of the first embodiment of the vessel sealing device inserted into a catheter sheath and in a portion of a patient's body;

FIG. 3 is a cross sectional view of the vessel sealing device of FIG. 2;

FIG. 4 is a partial cross sectional view of another embodiment of a vessel sealing device according to the present invention illustrating latching features on the shaft;

FIG. 5 illustrates the vessel sealing device of FIG. 3 percutaneously in a patient in a partial cross sectional view;

FIG. 6 illustrates the sealing device of FIG. 3 with the first and second sealing elements engaging the blood vessel;

FIG. 7 illustrates the sealing device of FIG. 3 after the shaft has been cut off;

FIG. 8 is a cross sectional view of another embodiment of a vessel sealing device with a tracer wire according to the present invention;

FIG. 9 is a perspective view of another embodiment of a vessel sealing device according to the present invention;

FIG. 10 is a partial view of one end of the vessel sealing device of FIG. 9, with the first and second portions of the rigid shaft rotated relative to one another;

FIG. 11 is a partial view of one end of the vessel sealing device of FIG. 9, with the first and second portions moved axially relative to one another; and

FIG. 12 is a partial view of one end of the vessel sealing device of FIG. 9, with the second sealing element present.

### DETAILED DESCRIPTION OF THE PREFERRED EMBODIMENTS

Reference will now be made in detail to the present preferred embodiment(s) of the invention, examples of which are illustrated in the accompanying drawings. Whenever possible, the same reference numerals will be used throughout the drawings to refer to the same or like parts.

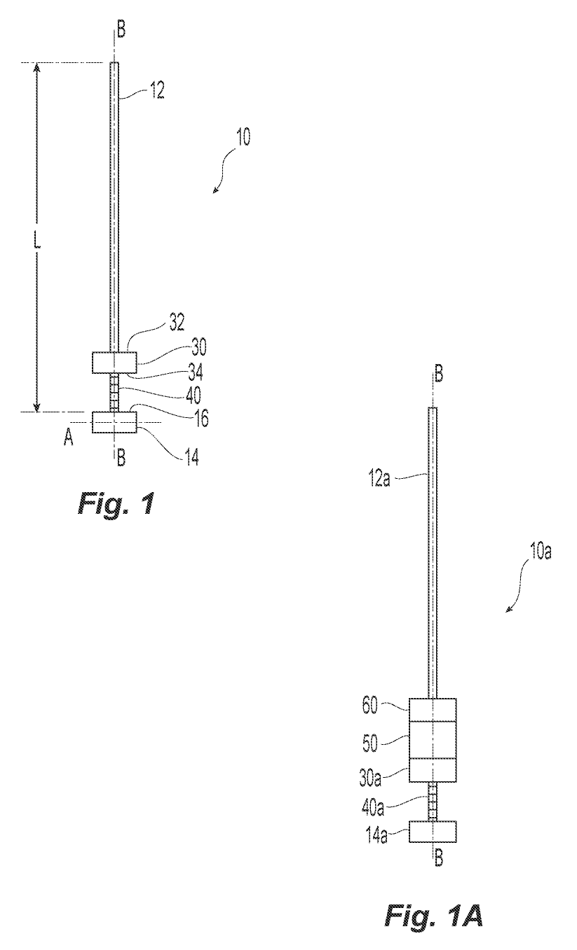

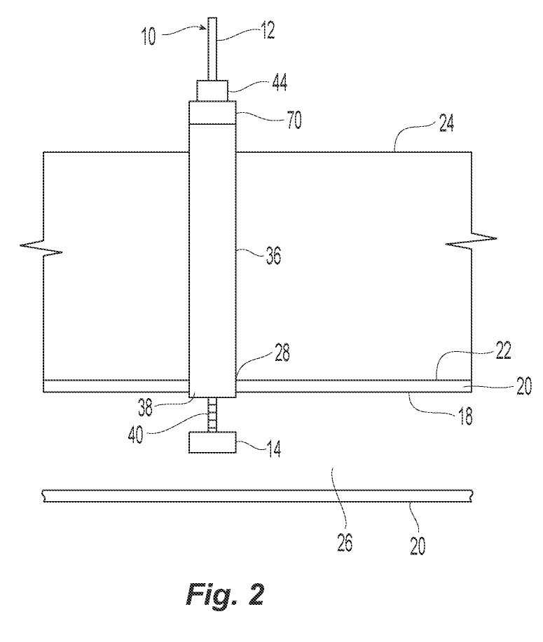

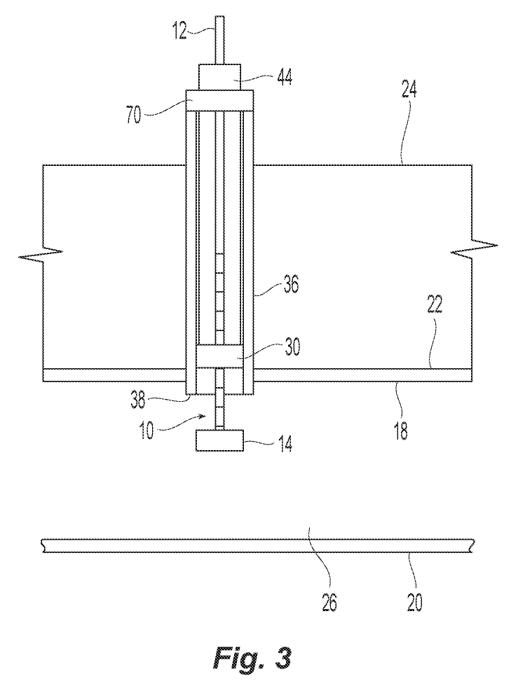

Referring to FIGS. 1, 2 and 3, a sealing device 10 is illustrated. The sealing device 10 includes a rigid shaft 12 having a first sealing element 14 attached thereto. The shaft 12 has a length L that extends outside any overlying tissue when the first element 14 is inserted into a lumen of a blood vessel. See, e.g., FIGS. 2 & 3. The first sealing element 14 has an upper surface 16 that engages the interior wall 18 of the blood vessel 20. The upper surface 16 is parallel to an axis A that extends through the first sealing element. The shaft 12 has a longitudinal axis B extending along the length thereof. First sealing element 14 is attached to the shaft 12 such that axis A of the first sealing element 14 is perpendicular to the longitudinal axis B of shaft 12. In one preferred embodiment, the shaft 12 and first sealing element 14 are a single, integral component, i.e., one singular piece. In another embodiment, the shaft 12 and first sealing element 14 are two separate components that are later fixedly attached to one another. They can be fixed to one another by adhesives, ultrasonic welding, etc.

The sealing device 10 also includes a second sealing element 30 that is slidingly movable along the rigid shaft 12 and relative to the first sealing element 14. Preferably the second sealing element 30 is movable along the rigid shaft 12 in only one direction, i.e., towards the first sealing element 14. The second sealing element 30 has an upper surface 32 and a lower surface 34, the lower surface engaging an outer surface 22 of the blood vessel 20 and also to cause the upper surface 16 of the first sealing element 14 to engage the inner surface 18 of the blood vessel 20. Preferably, the first sealing element 14, the second sealing element 30, and the rigid shaft are all made of a resorbable material such as a homo or co polymer of Lactide and/or Glycolide The sealing device also includes a pushing element 44 that engages the upper (or proximal) surface 32 of the second sealing element 30 to move the second sealing element 30 relative to the rigid shaft 12 and first sealing element 14.

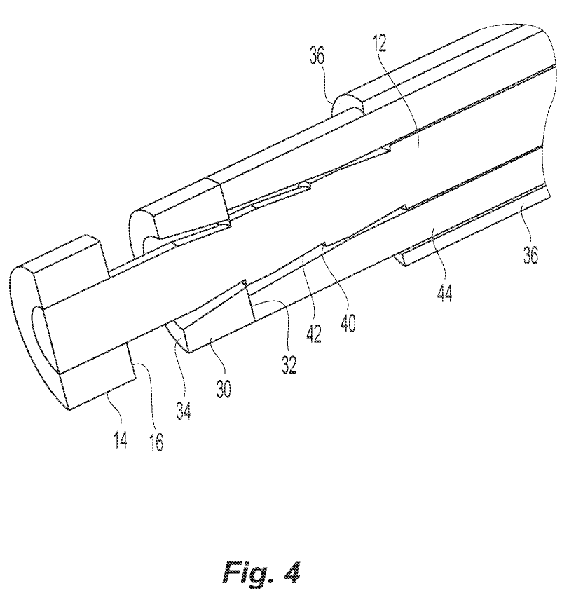

As better illustrated in FIG. 4, the rigid shaft 12 preferably includes a plurality of forward facing surfaces 40 that engage the upper surface 32 of the second sealing element 30 and prevents the second sealing element 30 from moving away from the first sealing element 14. Each of the forward facing surfaces 40 are generally perpendicular to the longitudinal axis B of the rigid shaft 12. Additionally, adjacent each of the forward facing surfaces 40 is a rear-ward sloped surface 42 that assists in maintaining the second sealing element 30 against the forward facing surfaces 40. Preferably, the forward facing surfaces 40 extend only along a bottom portion of the rigid shaft 12. Most preferably, the forward facing surfaces 40 extend along the bottom 15 mm of the rigid shaft 12.

In an alternative embodiment illustrated in FIG. 1A, sealing device 10a includes a rigid shaft 12a, a first sealing element 14a attached thereto, and a second sealing element 30a as in the first embodiment. However, sealing device 10a also preferably includes a collagen plug 50 disposed on a proximal side 32a of the second sealing element 30a. The collagen plug 50 preferably has the same diameter as the first sealing element 14 and the second sealing element 30a, but may have a larger or smaller diameter and still come within the scope of the present invention. The collagen plug 50 is not used to seal the opening in the blood vessel as discussed in more detail below, but rather to absorb any blood that may escape from the opening sealed by the first sealing element 14 and the second sealing element 30.

A restraining element 60 is used to engage an upper side of the collagen plug 50. The restraining element 60 frictionally engages the rigid shaft 12 to maintain the collagen plug 50 against the second sealing element 30. The restraining element 60 is preferably made of a homo or co polymer of Lactide and/or glycolide 1 mm thick. In the alternative embodiment, the pushing element 44 engages the restraining element 60 rather than the second sealing element 30 to move the second sealing element 30, the collagen plug 50 and the restraining element 60 toward the first sealing element 14.

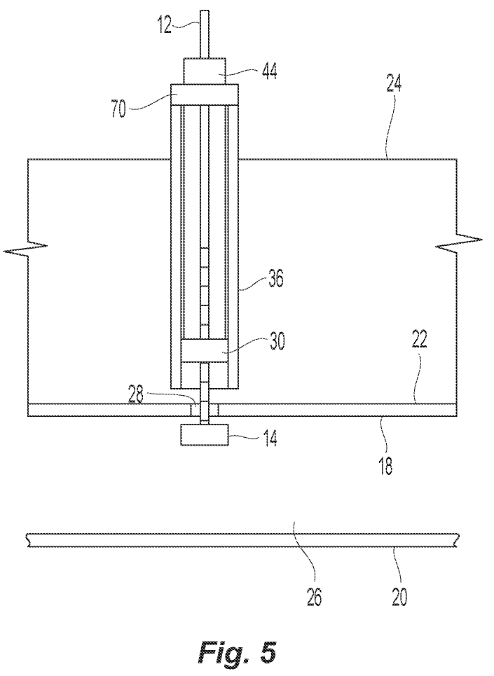

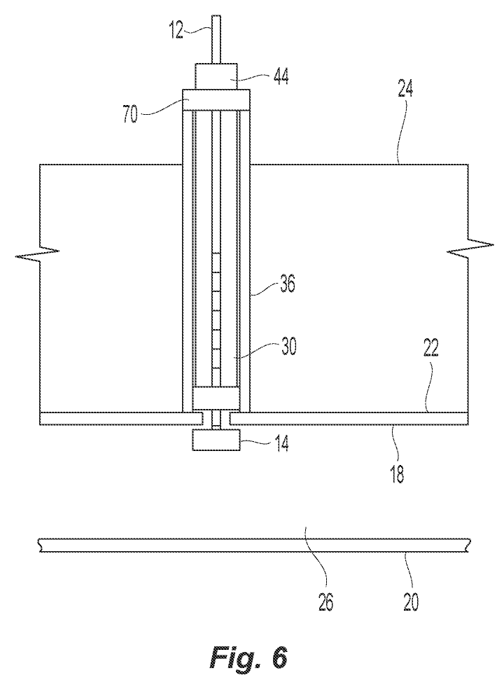

The operation of the sealing devices will now be explained with reference to FIGS. 2, 3, 5 and 6. As is known in the art, an opening is made through the overlying skin and tissue 24 and into the blood vessel 20 by way a catheter sheath 36. At least an end portion 38 of the catheter sheath 36 will be in the lumen 26 of the blood vessel 20. The sealing device 10 is inserted into the catheter sheath 36 through a hemostatic valve 70 such that the first sealing element 14 passes beyond a distal end 38 of the catheter sheath 36 and through an opening 28 in the blood vessel 20 and into the lumen 26 of the blood vessel 20. See FIGS. 2 & 3. The catheter sheath 36 is then pulled proximally to remove the end portion 38 of the catheter sheath 36 from the lumen 26 of the blood vessel 20 and also move the sealing device 10 in a proximal direction relative to the blood vessel 20. See FIG. 5. The opening 28 of the blood vessel 20 will then close around the rigid shaft 12 of the sealing device 10, causing the upper surface 16 of the first sealing element 14 to engage the interior wall 18 of the blood vessel 20. The rigid shaft 12 of the sealing device will then move relative to the catheter sheath 36, indicating that the first sealing element 14 has engaged the interior wall 18 of the blood vessel 20. The pushing element 44 is then moved distally relative to the rigid shaft 12 and catheter sheath 36 to move the second sealing element 30 toward the first sealing element 14 and the opening 28 in the blood vessel 20. See FIG. 6. The first and second sealing elements 14,30 pinch the blood vessel therebetween to seal the opening 28. The forward facing surfaces 40 prevent the second sealing element 30 from moving away from the blood vessel 20 or the opening 28. The catheter sheath 36 is then removed from the patient and the rigid shaft is appropriately trimmed so that it does not injure the patient while it is resorbed.

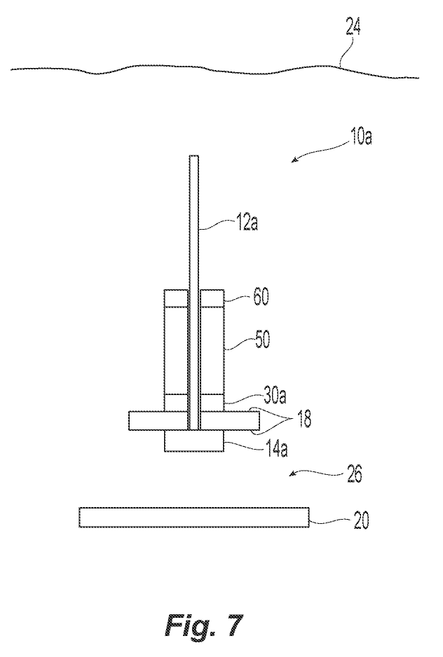

As illustrated in FIG. 7, the same process may be used with the sealing device 10a. Rather than the pushing element 44 pushing on the second sealing element 30a, it pushes on the restraining element 60, which in turn pushes on the collagen plug 50 and the second sealing element 30a. See FIG. 6. In this embodiment, the first and second sealing elements 14a, 30a seal the opening 28 in the blood vessel 20, but the collagen plug 50 absorbs any blood that may escape during the sealing the process. As noted above the restraining element frictionally engages the rigid shaft 12 to prevent the collagen plug 50 from moving away from the second sealing element 30.

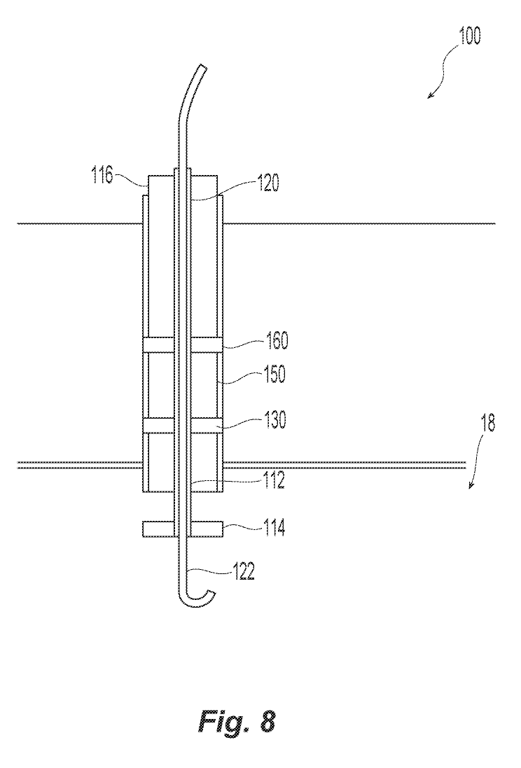

Another embodiment of a sealing device 100 is illustrated in FIG. 8. The sealing device 100 includes a rigid shaft 112, a first sealing element 114 attached thereto, and a second sealing element 130. As illustrated, sealing device 100 may also include a collagen plug 150 and a restraining element 160, although the collagen plug 150 and restraining element 160 are not necessary. The sealing device 100 also preferably includes a pushing element 116, which pushes on either the second sealing element 130 or the restraining element 160 as noted above. The rigid shaft 112 has an opening 120 extending therethrough and is configured to receive a tracer wire 122. Tracer wire 122 serves as a guide for the insertion of other devices in to vessel 118 in case any changes in the procedure are necessary prior to the sealing process. The diameter of opening 120 is small such that rapid clotting occurs after removal of the tracer wire 122, less than 0.5 mm for example.

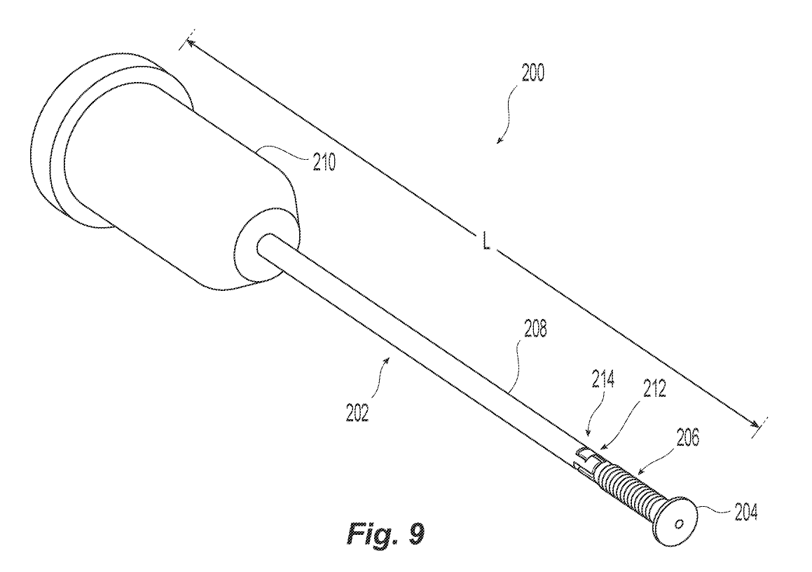

Another embodiment of a sealing device 200 is illustrated in FIGS. 9-12. The sealing device 200 includes a rigid shaft 202 having a first sealing element 204 attached thereto. In this embodiment, the rigid shaft 202 preferably has a first portion 206 and a second portion 208 and an overall length L that extends outside any overlying tissue when the first element 204 is inserted into a lumen of a blood vessel. Preferably, the first portion 206 is between 5 mm and 25 mm in length, although the length of either or both of the portions may be of any appropriate length depending on the patient and the use of the sealing device 200. While the first portion 206 is attached to the first sealing element 204 and the second portion 208 is attached to a handle 210. The first sealing element 204 is preferably the same as the first sealing element discussed above. Similarly, in one preferred embodiment, the first portion 206 and first sealing element 204 are a single, integral component, i.e., one singular piece. In another embodiment, the first portion 206 and first sealing element 204 are two separate components that are later fixedly attached to one another. They can be fixed to one another by adhesives, ultrasonic welding, etc.

At a first end 212 of the first portion 206 and also at a first end 214 of the second portion 208 are cooperating elements 216,218,220,222 that engage each other to secure the first and second portions 206,208 of the rigid shaft 202 to one another to allow for the sealing of the opening in a blood vessel. The sealing device 200 is preferably shipped with the first and second portions 206,208 engaging one another as illustrated in FIG. 9.

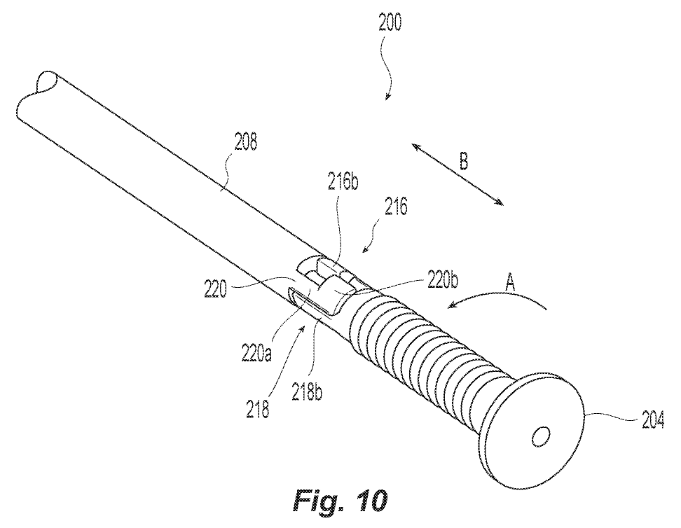

As can be seen in FIG. 10, where the first portion 206 is rotated 45° relative to second portion 208, each of the cooperating elements have two portions, for example, cooperating element 216 has a smaller neck region 216a and an extension portion 216b. The extension portion 216a extends from the neck region 216a in a generally orthogonal direction from the neck region to essentially form a “7.” Each of the other cooperating elements 218, 220, and 222 are similarly constructed. It should also be noted that the outer surface of cooperating elements 216,218,220,222 are an extension of the other portions of the rigid shaft 202, causing the rigid shaft to be essentially continuous along its length when assembled as shown in FIG. 9. In FIG. 10, the cooperating elements 216 and 218 of first portion 206 have been disengaged from the cooperating elements 220 and 222 of the second portion 208 by rotating the two portions 206,208 relative to one another. Again referring to FIG. 10, if the first portion 206 is rotated in the direction of arrow A, the extension portion 216b of cooperating element 216 will rotate towards the neck region 220a of cooperating element 220 locking cooperating elements 216 and 220 together and prevent the first and second portions from moving axially relatively to one another as illustrated by arrow B. On the bottom side of the sealing device 200 as illustrated in FIG. 10, the cooperating elements 218 and 222 would also engage one another.

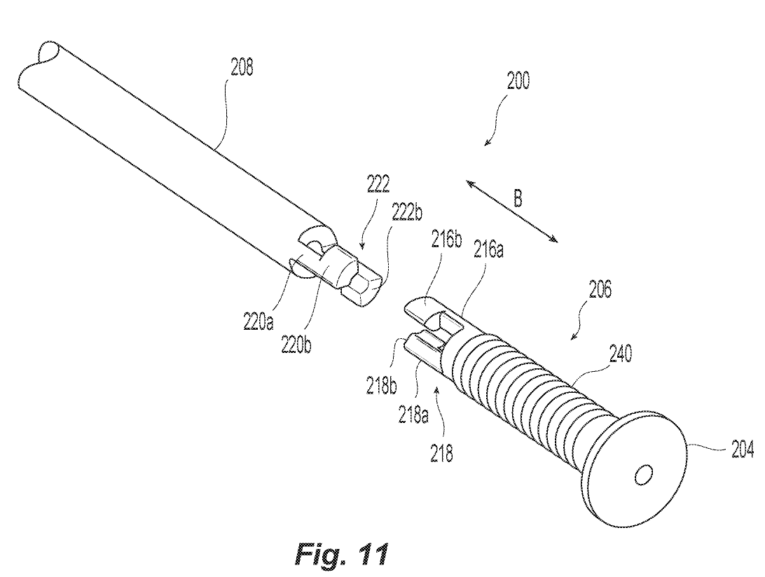

If the first and second portions 206, 208 are in the positions as illustrated in FIG. 10 and are then pulled away from one another (as illustrated by arrow B), the two portions 206, 208 will disengage from one another, as illustrated in FIG. 11. As can be seen in FIG. 11, the cooperating elements 216, 218 of the first portion 206 (and particularly extension portion 216b) pass between the two cooperating elements 218,220 of the second portion 208 (on opposite sides thereof).

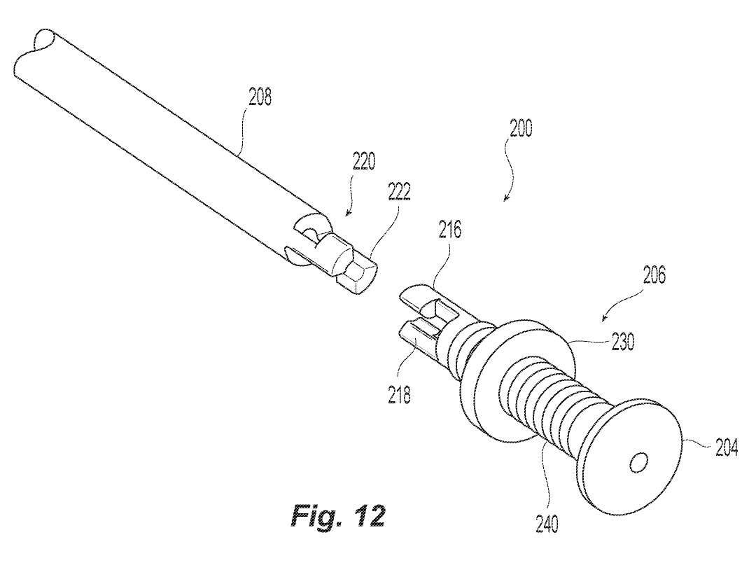

FIG. 12 shows sealing device 200 with the second sealing element 230 disposed on first portion 206 and engaging the surfaces 240 that prevent the second sealing element 230 from moving relative to the first portion 206 after the openings in the blood vessel.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed is:

1. A sealing device for sealing an opening in the wall of a blood vessel, the blood vessel having an interior wall surface, and exterior wall surface, and a lumen, the sealing device comprising:
a first sealing element for placing inside the lumen of the blood vessel and to engage the interior wall surface thereof;
a rigid shaft fixedly attached to the first sealing element in a predetermined configuration, the rigid shaft having a length sufficient to extend through the opening of the blood vessel and at least a portion of any overlying tissue; and
a second sealing element, the second sealing element slidingly movable relative to the first sealing element in only one direction along the rigid shaft to engage the outside surface of the blood vessel and position the first sealing element against the interior surface of the blood vessel to seal the opening in the blood vessel.

2. The sealing device according to claim 1, wherein the first sealing element, the second sealing element, and rigid shaft are made of a resorb able material.

3. The sealing device according to claim 1, wherein the first sealing element and rigid shaft are one integral element.

4. The sealing device according to claim 1, wherein the second element is rigid.

5. The sealing device according to claim 1, wherein the rigid shaft and the first sealing element are one integral element.

6. The sealing device according to claim 1, wherein the first sealing element has an upper surface to engage the interior wall of the blood vessel and an axis parallel thereto, and the rigid shaft has a longitudinal axis therealong, and wherein the first sealing element axis and the longitudinal axis of the rigid shaft are perpendicular to one another.

7. The sealing device according to claim 1, further comprising a collagen plug slidingly engaging the rigid shaft, the collagen plug disposed on a proximal side of the second sealing element.

8. The sealing device according to claim 7, further comprising a restraining element, the restraining element slidingly engaging the rigid shaft and holding the collagen plug against the second sealing element.

9. The sealing device according to claim 1, wherein the rigid shaft includes a plurality of forward facing surfaces, the forward facing surfaces being generally perpendicular to a longitudinal axis extending along the length of the rigid shaft, the forward facing surfaces engaging a rearward facing surface of the second sealing element to retain the second sealing element relative to the rigid shaft.

10. The sealing device according to claim 9, wherein the plurality of forward facing surfaces extend only along a portion of the length of the rigid shaft.

11. The sealing device according to claim 1, further comprising a pushing element, the pushing element engaging a proximal surface of the second sealing element to slidingly move the second sealing element along the rigid shaft to engage the outer surface of the blood vessel.

12. The sealing device according to claim 8, further comprising a pushing element, the pushing element engaging a proximal surface of the restraining element to slidingly move the restraining element, the collagen plug, and the second sealing element along the rigid shaft to engage the outer surface of the blood vessel.

13. The sealing device according to claim 1, wherein the rigid shaft comprises a first portion and a second portion, the first portion being fixedly attached to the first sealing element and the second portion being connected to a handle, the second portion of the rigid shaft removably engages the first portion of the rigid shaft.

14. The sealing device according to claim 13, wherein the first and second portions are rotated relative to one another to lock the first and second portions to one another.

15. The sealing device according to claim 14, wherein the first and second portions must be moved axially relative to one another to disengage one another.

16. The sealing device according to claim 13, wherein the first portion is shorter than the second portion.

17. A method of sealing an opening in the wall of a blood vessel, the blood vessel having an interior wall surface, and exterior wall surface, and a lumen, the method comprising the steps of:
providing a sealing device comprising a first sealing element, a rigid shaft fixedly attached to the first sealing element in a predetermined configuration, the rigid shaft having a length sufficient to extend through the opening of the blood vessel and at least a portion of any overlying tissue, and a second sealing element, the second sealing element slidingly movable relative to the first sealing element in only one direction along the rigid shaft;
providing an elongated sheath, the sheath capable of receiving the sealing device into an interior opening extending along the sheath;
inserting the sheath into the opening in the wall of the blood vessel;
inserting the sealing device into the opening in the wall of the blood vessel through the sheath such that at least the first sealing element extends beyond a distal end of the sheath and is disposed in the lumen of the blood vessel;
moving the sheath in a proximal direction and out of the blood vessel opening such that the first sealing element engages at least a portion of the interior wall surface of the blood vessel;
slidingly moving the second element along the rigid shaft and towards the first sealing element such that the second element engages the exterior wall surface of the blood vessel, thereby pinching the blood vessel between the first and second sealing elements to seal the opening in the blood vessel; and
removing the sheath from the sealing device.

18. The method according to claim 17, wherein the sealing device is inserted into the sheath prior to the sheath being inserted into the opening of the blood vessel.

19. The method according to claim 17, wherein the sealing device is inserted into the sheath after the sheath has been inserted into the opening of the blood vessel.

20. The method according to claim 17, wherein the sealing device further comprises a pushing element, the pushing element configured to be disposed within the sheath and engage the second sealing element, whereby pressure exerted on the pushing element moves the second sealing element relative to the rigid shaft towards the first sealing element.

21. The method according to claim 17, further comprising the step of removing at least a portion of the rigid shaft so that a proximal end of the rigid shaft remains within the overlying tissue.

22. The method according to claim 17, wherein the first sealing element, the second sealing element, and rigid shaft are made of a resorb able material.

23. The method according to claim 17, wherein the first sealing element and rigid shaft are one integral element.

24. The method according to claim 17, wherein the second element is rigid.

25. The method according to claim 17, wherein the rigid shaft and the first sealing element are one integral element.

26. The method according to claim 17, wherein the first sealing element has an upper surface to engage the interior wall of the blood vessel and an axis parallel thereto, and the rigid shaft has a longitudinal axis therealong, and wherein the first sealing element axis and the longitudinal axis of the rigid shaft are perpendicular to one another.

27. The method according to claim 17, further comprising a collagen plug slidingly engaging the rigid shaft, the collagen plug disposed on a proximal side of the second sealing element.

28. The method according to claim 27, further comprising a restraining element, the restraining element slidingly engaging the rigid shaft and holding the collagen plug against the second sealing element.

29. The method according to claim 17, wherein the rigid shaft includes a plurality of forward facing surfaces, the forward facing surfaces being generally perpendicular to a longitudinal axis extending along the length of the rigid shaft, the forward facing surfaces engaging a rearward facing surface of the second sealing element to retain the second sealing element relative to the rigid shaft.

30. The method according to claim 17, wherein the plurality of forward facing surfaces extend only along a portion of the length of the rigid shaft.

31. The method according to claim 17, further comprising a pushing element, the pushing element engaging a proximal surface of the second sealing element to slidingly move the second sealing element along the rigid shaft to engage the outer surface of the blood vessel.

32. The method according to claim 17, wherein the rigid shaft comprises a first portion and a second portion, the first portion being fixedly attached to the first sealing element and the second portion being connected to a handle, the second portion of the rigid shaft removably engages the first portion of the rigid shaft.
